(19) United States
(12) Patent Application Publication
(10) Pub. No.: US 2009/0216174 A1
(43) Pub. Date: Aug. 27, 2009

(54) DOUBLE LUMEN DIALYSIS CATHETER WITH CLOSEABLE ARTERIAL DISTAL PORT

(76) Inventor: Mahase Nardeo, Collegeville, PA (US)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/388,589

(22) Filed: Feb. 19, 2009

Related U.S. Application Data
(60) Provisional application No. 61/066,531, filed on Feb. 21, 2008.

(51) Int. Cl. A61M 25/00

(52) U.S. Cl. 604/6.16

(57) ABSTRACT

A dialysis catheter which includes a first tube having a first tube distal end with an intake port and a second tube that extends parallel to and is connected to the first tube, with the second tube having a second tube distal end with a discharge port is provided. A cover is located on the first tube that it movable from a first position, in which it blocks the intake port, to a second position, which the intake port is unblocked. A longitudinally extending control wire hole is defined in a wall of the first tube. A control wire extends through the hole, with the control wire having a first end that is fixed to the cover. The second end of the control wire is connected to a switch that is moveable in order to move the cover between the first and second positions. This arrangement helps to prevent blockage of the arterial intake port due to thrombosis and fibrin buildup.

### CROSS REFERENCE TO RELATED APPLICATION(S)

This application claims the benefit of U.S. provisional application No. 61/066,531, field Feb. 21, 2008, which is incorporated by reference as if fully set forth.

### BACKGROUND

The present invention relates to the field of dialysis catheters for insertion into body vessels for the purpose of dialyzing patients with kidney failure. Typically, dialysis catheters are double lumen catheters having proximal and distal ends with lumens extending between the two ends. The distal end is placed where there is a high flow of blood, typically near the heart. The high blood flow enables a sufficient volume of blood to be processed through the catheter and cleaned by a dialysis machine. The proximal end is typically placed on the chest for easy access to connect blood lines from the dialysis machine.

A typical medical procedure for implanting a catheter when accessing the jugular vein involves a small incision in the skin of the neck over the jugular vein. Subsequently, the vessel is dilated to allow the insertion of an introducer sheath. The sub-cutaneous tissue under the skin, adjacent to the insertion site, is tunneled with a dilator, thus allowing the passage and anchoring of the catheter right above the clavicle. After insertion of the catheter, the sheath is removed by peeling it apart.

Dialysis catheters vary considerably from one manufacturer to another. However, one common requirement for virtually all dialysis catheters is open-ended distal ends for the removal of blood from the blood vessel to the dialysis machine and the return of the processed blood from the dialysis machine to the blood vessel. One known problem is that the open-ended arterial intake port is prone to blockage by thrombosis and fibrin buildup, thus preventing or reducing the flow considerably. When this happens and the blood flow cannot be restored, doctors are forced to remove the catheter and replace it with a new catheter. This process is very costly and induces additional trauma to the patient. More importantly, it also puts the patient at further risks for infection during the catheter exchange procedure.

### SUMMARY

In one aspect, a dialysis catheter is provided having a first tube with a first tube distal end having an intake port. A second tube extends parallel to and is connected to the first tube, and includes a second tube distal end with a discharge port. A cover is located on the first tube that is movable from a first position, in which it blocks the intake port, to a second position, in which the intake port is unblocked. A longitudinally extending control wire hole is defined through a wall of the first tube. A control wire extends through the hole. The control wire has a first end that is fixed to the cover and a second end. A switch is connected to the second end of the control wire that is movable in order to move the cover between the first and second positions.

Preferably, during use in dialysis procedures, the arterial intake port is closed at all times until the patient is ready for dialysis treatment. By keeping the arterial intake closed by the cover, buildup of fibrin or thrombosis is kept on the outside of the port. When dialyzing the patient, the switch is moved so that the cover is in the second, open position where the intake port is unblocked, which also allows for the clearing of any buildup on the intake port. Once the dialysis treatment is completed, the switch is moved back so that the cover is in the first position, covering the intake port.

Preferably after dialyzing the patient, each of the lumens defined by the first and second tubes is primed with anti-coagulent medication, typically Heparin, to prevent blood from clotting the lumens. In the known prior devices, the Heparin leaks out the open-ended ports, which leads to clotting of the lumens. The closed arterial port provided by the present invention reduces the chances of Heparin leaking out of the intake port, thus acting further to prevent blockage of the arterial lumen. Most blood flow blockage problems are associated with the arterial intake lumens, although a similar cover could also be provided for the venal discharge port from the second tube, if desired.

### BRIEF DESCRIPTION OF THE DRAWINGS

The foregoing summary as well as the following detailed description will be better understood when read in conjunction with the appended drawings where a preferred embodiment of the invention is shown. In the drawings:

FIG. 1 is a side elevational view of a dialysis catheter in accordance with the present invention.

FIG. 2 is a top cross-sectional view of the distal end of the catheter of FIG. 1, taken along lines 2-2 in FIG. 1.

FIG. 3 is a cross-sectional view of the distal end of the catheter taken along lines 3-3 in FIG. 2.

FIG. 4 is a cross-sectional view taken along lines 4-4 in FIG. 3.

FIG. 5 is an enlarged cross-sectional view similar to the cross-section of the catheter shown in FIG. 4.

FIG. 6 is a perspective view of the hub portion of the dialysis catheter of FIG. 1.

FIG. 7 is a cross-sectional view through the hub shown in FIG. 6.

FIG. 8 is a perspective view showing the arterial intake port and cover in an enlarged view.

FIG. 8A is an enlarged perspective view showing the intake port with the cover in an open position.

FIG. 8B is an enlarged top view showing the cover in an open position.

FIG. 8C is an enlarged front view of the intake port showing the cover in the open position.

FIG. 8D is an enlarged side view showing the cover in an open position.

FIG. 9 is a perspective view showing the distal end of the catheter with the arterial intake port cover in a closed position and the venal tube discharge port.

FIG. 10 is a perspective view similar to FIG. 9 showing the cover in the open position.

### DETAILED DESCRIPTION OF THE PREFERRED EMBODIMENTS

Certain terminology is used in the following detailed description for convenience only and is not considered limiting. The words “upper” and “lower” designate direction in the drawings to which reference is made. Additionally, the terms “proximal” and “distal” refer to the dialysis catheter based upon the portion which would be handled by a doctor or nurse setting the catheter in a patient for dialysis treatment. Additionally, the terms “a” and “one” are defined as including one or more of the referenced items unless specifically noted.

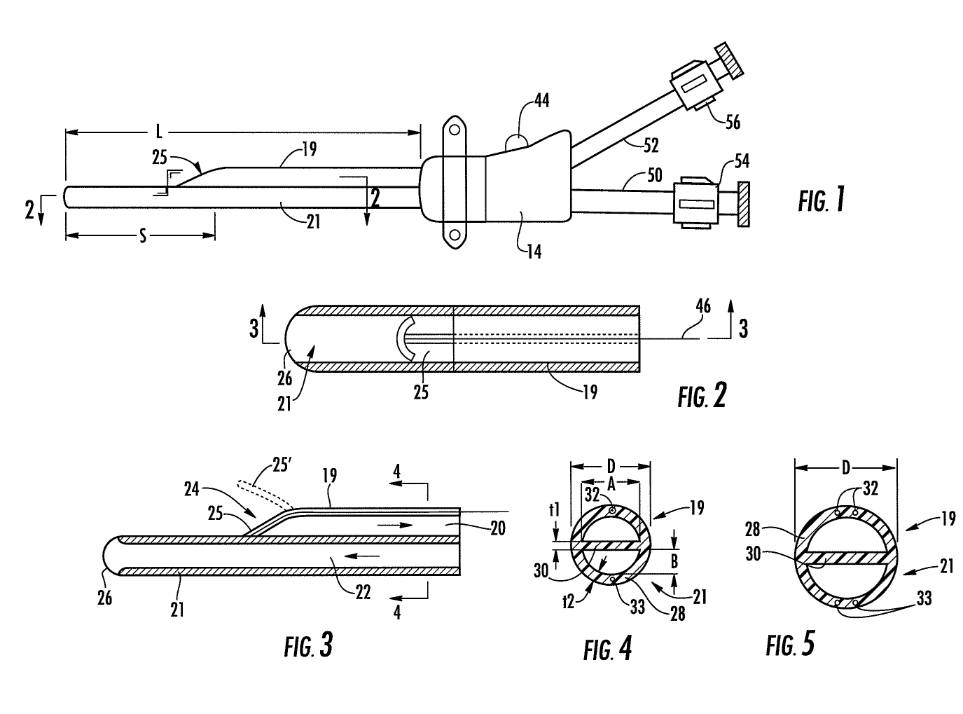

Referring to FIG. 1, a dialysis catheter 10 in accordance with a preferred embodiment of the present invention is shown. The dialysis catheter 10 includes a first tube 19 having a first tube distal end with an intake port 24. A second tube 21 extends parallel to and is connected to the first tube 19. The second tube 21 has a second tube distal end with a discharge port 26.

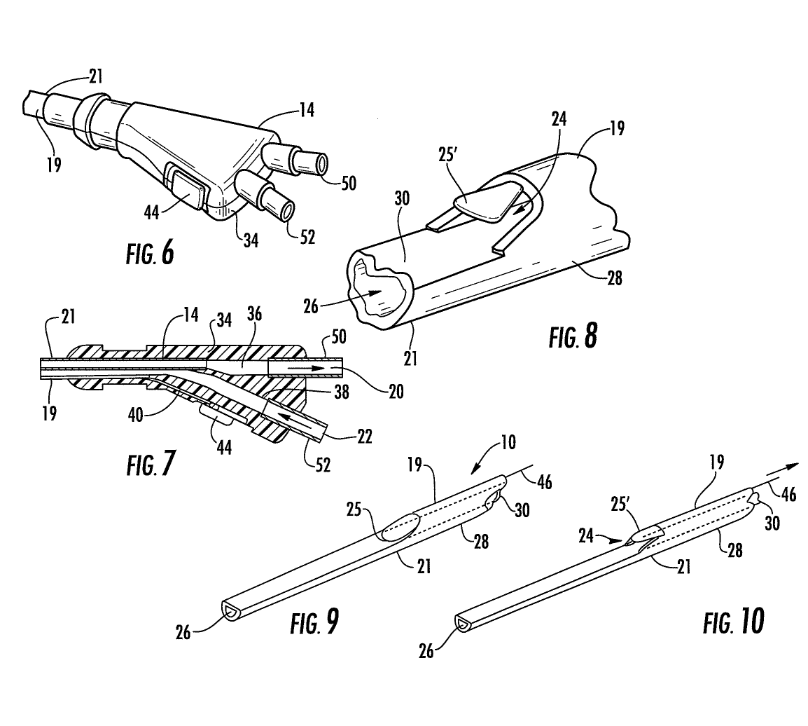

In the preferred embodiment, the first and second tubes 19, 21 are formed as a unitary dual tube having a generally circular outer wall 28 and an inner dividing wall 30, as shown in FIG. 4. The outer wall has a diameter D which is preferably 0.192 inches. The inner dividing wall 28 preferably has a length A of approximately 0.132 inches. This divides the interior into two semi-circular shaped profiles, each having a height B of approximately 0.06 inches. The dividing wall 30 preferably has a thickness t₁ of about 0.012 inches and the outer wall 28 has a thickness t₂ of approximately 0.03 inches. Preferably, longitudinally extending holes 32, 33 are defined in the outer wall 28. The holes 32, 33 preferably have a diameter of 0.021 inches. Alternatively, as shown in FIG. 5, the outer wall 28 can have two holes 32 defined in the top portion and two holes 33 defined in the bottom portion. Otherwise the dimensions of the unitary dual tube are the same as discussed above in connection with FIG. 4.

Preferably, the dual tube 19, 21 is made of a polyurethane or silicone material. However, other suitable medical grade materials may be utilized.

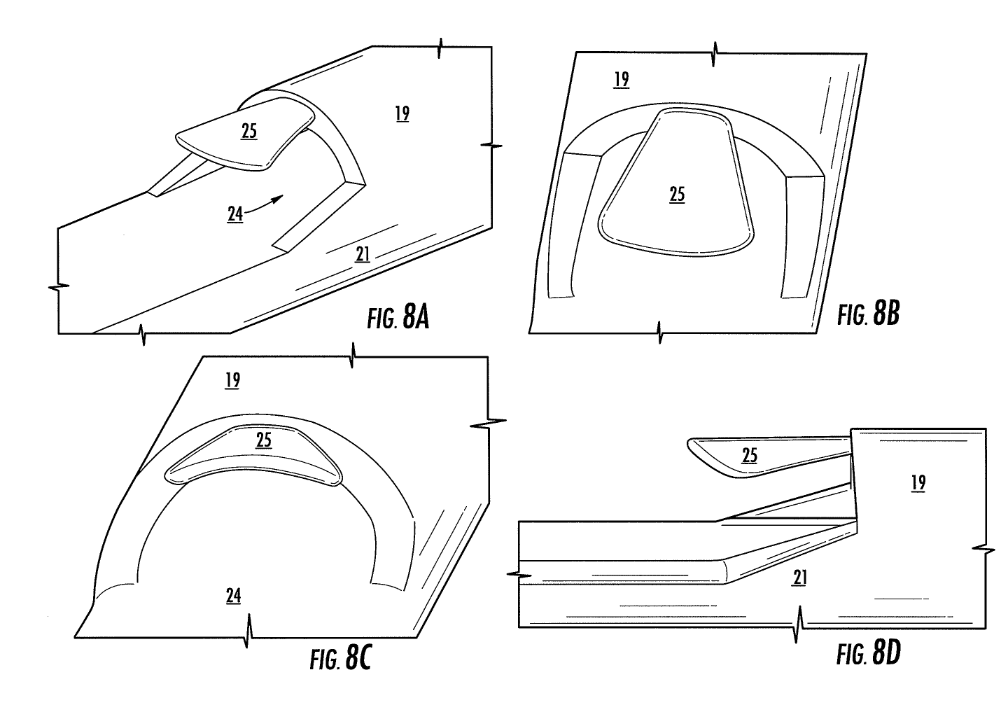

Referring to FIGS. 2, 3, 8 and 8A-8D, a cover 25 is located on the first tube 19 that is movable from a first position, shown in FIGS. 2, 3 and 9, in which the cover 25 blocks the intake port 24, and a second position, shown in dashed lines in FIG. 3 and shown in FIGS. 8, 8A-8D and 10, in which the cover 25 is in a second position in which the intake port 24 is unblocked. The cover 25 is preferably also made of a flexible polymeric material such as silicone or polyurethane. The cover 25 is connected to the outer wall 28 of the first tube via a living hinge formed as part of the cover material or via a flexible material which is connected between the cover 25 and the first tube in proximity to the top of the first tube in the area of the intake port 24.

The longitudinally extending hole 32 in the outer wall 28 of the first tube in proximity to the top thereof, as shown in FIG. 4, is used for a control wire 46 that extends through the hole 32. The control wire 46 has a first end that is fixed to the cover 25 and a second end. The second end of the control wire 32 is connected to a switch 44, best shown in FIGS. 6 and 7, that is movable in order to move the cover 25 between the first and second positions, shown best in FIGS. 9 and 10 as 25 and 25′, respectively.

Referring to FIGS. 1, 6 and 7, the catheter 10 further comprises a hub 14, and the first and second tubes 19, 21 are connected to the hub 14. The hub 14 is preferably formed from a two-piece molded body 34, which can be formed in two halves split along the longitudinal center plane, shown in FIG. 7. The hub 14 includes first and second passages 36, 38 that extend from the first and second tubes 19, 21 to a proximal side of the hub 14. A proximal arterial tube 50 is connected to the first passage 36 and extends from the proximal side of the hub outwardly for connection to a dialysis machine. A proximal venal tube 52 is connected to the second passage 38 and extends from the proximal side of the hub 14 to provide the return path connection from the dialysis machine. The first tube 19, the first passage 36 and the proximal arterial tube 50 define an arterial lumen 20 that is adapted to extend from the intake port 24 within a patient's blood vessel to a dialysis machine. The second tube 21, the second passage 38 and the proximal venal tube 52 define a venal lumen 22 that extends back from the dialysis machine to the discharge port within the patient's blood vessel in order to return treated blood to the patient. The dialysis catheter 10 has as small a diameter as possible which can still provide a desired blood flow rate of 500 ml/minute.

As shown in FIG. 7, preferably a third passage 40 is defined in the hub 14 for the actuator wire 46 and extends from a switch recess 42, where the switch 44 is slidably mounted, to a position aligned with the longitudinally extending control wire hole 32 in the outer wall 28 of the first tube 19.

The second longitudinally extending hole 33, shown in FIG. 4, can be used for a reinforcing wire, if desired. This helps to prevent kinking of the thin-walled catheter as it is being inserted into the blood vessel. Alternatively, the hole 33 can remain unused.

As a further alternative, when the unitary dual tube shown in FIG. 5 is utilized, the control wire 46 can be formed as a loop and extend through both of the control wire holes 32 provided between the cover 25 and the switch 44. One or both holes 33 can be utilized for reinforcing wires on the opposite side of the outer wall 28 from the guide wire holes 32.

Referring again to FIG. 1, preferably Luer connectors 54, 56 are provided on the ends of the proximal arterial and venal tubes 50, 52. Alternatively, other types of connectors can be utilized.

In the preferred embodiment, the hub 14 is formed as a molded part or parts, and is preferably formed in two halves that can be joined together to connect the tubes 19, 21, 50 and 52 to the passages 36 and 38. By forming the hub 14 as two molded halves 34, the tubes can be accurately set in position and can be sealed in place by bonding or adhering the two halves together. The mating plane of one half is shown in FIG. 7 and the opposite mating plane would a mirror image thereof. Once joined together, the catheter assembly 10 can be pressure checked to ensure that there is no leakage between the arterial lumen 20 and venal lumen 22. Alternatively, the hub can be molded in one piece around the tubes using removable mandrels to define the passages 36, 38, 40 through the hub 14.

In use, the catheter 10 is inserted into a patient's blood vessel with the cover 25 in the closed position. When dialysis is to be initiated, the switch 44 is utilized to move the cover 25 to the second, open position such that the intake port 24 is unblocked. Blood is withdrawn from the patient via the arterial lumen defined by the first tube 19, first passage 36 and the proximal arterial tube 50 where it is drawn into the dialysis machine and treated. The treated blood is returned via the venal lumen 22 defined by the proximal venal tube 52, the second passage 38 and the second tube 21. At the end of the dialysis treatment, the switch 44 is moved to return the cover 25 to the closed position where the arterial port 24 is blocked. Each lumen is then primed with anti-coagulant medication, preferably Heparin to prevent blood from clotting the lumens. The closed arterial port reduces the chance of Heparin leaking out of the port and therefore prevents blockage of the arterial lumen. As most blood flow blockage problems are associated with the arterial lumen in practice, a cover has only been shown as being provided for the arterial port 24. However, it is also possible within the scope of the present invention to provide a second cover for the discharge port 26 from the second tube 21, if desired.

Referring again to FIG. 1, in the preferred embodiment the dimension L for the overall length of the distal tubes 19, 21 can range from about 10 inches to about 18 inches. Preferably, the distance S, which is the offset of the arterial port 24 from the venal port 26 is approximately one inch.

Those skilled in the art will understand that the cover 25 does not need to entirely close the arterial port 24, but should generally cover a majority of the arterial port opening.

While the preferred embodiment of the invention has been described, the invention is not limited to the specific arrangement provided. Rather, the scope of the invention is defined by the appended claims.

What is claimed is:

1. A dialysis catheter comprising:
a first tube having a first tube distal end with an intake port,
a second tube, extending parallel to and connected to the first tube, the second tube having a second tube distal end with a discharge port,
a cover located on the first tube that is movable from a first position, in which it blocks the intake port, to a second position, in which the intake port is unblocked,
a longitudinally extending control wire hole defined in a wall of the first tube, and a control wire extending through the hole, the control wire having a first end that is fixed to the cover, and a second end, and
a switch connected to the second end of the control wire that is movable in order to move the cover between the first and second positions.

2. The dialysis catheter of claim 1, wherein the cover is connected to the wall of the first tube via a living hinge.

3. The dialysis catheter of claim 1, wherein the cover is connected to the wall of the first tube via a flexible member.

4. The dialysis catheter of claim 1, further comprising a hub, wherein the first and second tubes are connected to the hub, and the hub includes first and second passages that extend from the first and second tubes to a proximal side thereof, and a proximal arterial tube is connected to the first passage and extends from the proximal side of the hub, and a proximal venal tube is connected to the second passage and extends from the proximal side of the hub, the first tube, the first passage and the proximal arterial tube defining an arterial lumen, and the second tube, the second passage and the proximal venal tube defining a venal lumen.

5. The dialysis catheter of claim 4, wherein the first and second tubes are formed as a unitary dual tube having a generally circular outer wall and an inner dividing wall.

6. The dialysis catheter of claim 5, wherein the unitary dual tube is formed of a polyurethane or silicone material.

7. The dialysis catheter of claim 5, wherein the control wire hole is located in the outer wall on a first tube side of the unitary dual tube, and a second longitudinally extending hole is located in the outer wall on an opposite side from the control wire hole for a reinforcing wire.

8. The dialysis catheter of claim 4, wherein the switch is connected to the hub, and a third passage is defined in the hub for the guide wire.

9. The dialysis catheter of claim 8, wherein the hub is a molded polymeric part.

10. The dialysis catheter of claim 4, further comprising Luer connectors on the proximal arterial and venal tubes.

11. The dialysis catheter of claim 4, wherein the hub is formed as two halves that are joined together to connect the tubes to the passages.

12. The dialysis catheter of claim 1, wherein the control wire comprises stainless steel, Nylon or PEEK.
